(19) United States
(12) Patent Application Publication
(10) Pub. No.: US 2002/0009709 A1
(43) Pub. Date: Jan. 24, 2002

(54) METHOD FOR MEASURING THYROGLOBULIN

(75) Inventors: RYOJI KATO, NAGANO (JP); MASAYUKI MARUYAMA, NAGANO (JP); KENJI NAKAMURA, HYOUGO (JP); KAYOKO SHIMIZU, HYOUGO (JP); SHINJI SATOMURA, OSAKA (JP)

Correspondence Address: ARMSTRONG,WESTERMAN, HATTORI,, MCLELAND & NAUGHTON, LLP, 1725 K STREET, NW, SUITE 1000, WASHINGTON, DC, 20006, US

(73) Assignee: WAKO PURE CHEMICAL INDUSTRIES, LTD., OSAKA (JP)

(21) Appl. No.: 09/340,196

(22) Filed: Jun. 28, 1999

(30) Foreign Application Priority Data
Jun. 30, 1998 (JP) JP10-199794

(51) Int. Cl. C12Q 1/00

(52) U.S. Cl. 435/4

(57) ABSTRACT

A method for measuring thyroglobulin(s), comprising using each one or more kinds of proteins capable of binding to a constant region of thyroglobulin(s) and proteins capable of specifically binding to a specific sugar chain structure of thyroglobulin(s) having the specific sugar chain structure, and the method of determining a malignancy of thyroid tumor, a reagent thereof using the obtained by the method for measuring thyroglobulin(s).

### BACKGROUND OF THE INVENTION

The present invention relates to a method for measuring thyroglobulin (hereinafter abbreviated as Tg), more particularly a method for measuring Tg(s) having a specific sugar chain structure, and further relates to a method for determining malignancy of thyroid carcinoma based upon an amount of Tg(s) having a specific sugar chain structure.

It has been known that Tg(s) is a sugar protein of molecular weight of 660,000 composed of a dimer of subunits having molecular weight of 330,000, which is secreted from a thyroid gland and that its content in cells or blood is increased in benign or malignant thyroid gland diseases. For this reason, the measurement of Tg(s) in blood has been used in progress observation after an operation on thyroid tumor. However, it is not possible to determine whether the thyroid tumor is benign or malignant on the basis of the Tg(s) content.

Thus, determination of malignancy of thyroid tumor has been conducted by sonography, punch aspiration biopsy, etc. However, there have been such problems that in the sonography, highly skilled diagnosis technique is required and in the punch aspiration biopsy, several days are required in the determination when culture the collected cells, and on the other hand highly skilled technique is required when the determination is conducted by observation of cells collected. Further, in the punch aspiration biopsy, there is also such a problem that differentiation of follicular adenoma from follicular carcinoma is difficult.

Further, it has been tried that benign or malignant of thyroid tumor is determined by specifying a sugar chain on the surface of the cell with the use of protein recognizing a sugar such as a lectin, and as the result, it has been found that there is observed a difference in sugar chain structures between benign and malignant thyroid carcinoma. In this method, however, each cell collected is reacted with a labeled protein recognizing a sugar chain and then an amount of the labeled material is confirmed under microscope, and thus this method is accompanied with such problems that skilled technique is required for the test and quantitative evaluation of the test result is difficult.

On the other hand, purified Tg(s) have been obtained from benign cell and malignant carcinoma cell and the structures of their sugar chains have been analyzed to find that the structures of sugar chains in the cells are different between the benign cell and the malignant carcinoma cell. However, also in this method, there is such a problem that a long time is required for the purification of Tg(s) from the cells collected and for the analysis of the structures of the sugar chains.

### BRIEF SUMMARY OF THE INVENTION

Under the situation as mentioned above, the problem to be solved by the present invention is to provide a method for easily and simply measuring various kinds of Tg(s) in samples originated from a living body, to provide a method for determining malignancy of thyroid tumor on the basis of the measured result and to provide a reagent for this purpose.

The present invention has been accomplished for the purpose of solving those problems as mentioned above, and it relates to as follows;

(1) A method for measuring thyroglobulin(s), comprising using each one or more kinds of proteins capable of binding to a constant region of thyroglobulin(s) and proteins capable of specifically binding to a specific sugar chain structure of thyroglobulin(s) having the specific sugar chain structure.

(2) A reagent for measuring thyroglobulin(s), which comprises each one or more kinds of proteins capable of binding to a constant region of thyroglobulin(s) and proteins capable of specifically binding to a specific sugar chain structure of thyroglobulin(s) having the specific sugar chain structure.

(3) A reagent for determining malignancy of thyroid tumor, which comprises each one or more kinds of proteins capable of binding to a constant region of thyroglobulin(s) and proteins capable of specifically binding to a specific sugar chain structure of thyroglobulin(s) having the specific sugar chain structure.

(4) A method of determining a malignancy of thyroid tumor, which comprises measuring one or more of thyroglobulins having a specific sugar chain structures present in a sample originated from a living body and determining the malignancy of thyroid tumor on the basis of the amounts of the thyroglobulins.

(5) A method of determining a malignancy of thyroid tumor, which comprises separating thyroglobulins from one another present in a sample originated from a living body on the basis of the difference between or among their sugar chain structures, measuring the proportion of one or more of thyroglobulin(s) having a specific sugar chain structure, related to the total amount of thyroglobulin(s), and determining the malignancy of thyroid tumor on the basis of a result of the measurement.

### BRIEF DESCRIPTION OF THE DRAWINGS

FIG. 1 shows a ratio (%) of Tg(s) not bound to Concanavalin A related to the total Tg(s) in various kinds of thyroid tissue extracts obtained in Example 1.

FIG. 2 shows a ratio (%) of Tg(s) not bound to Ricinus communis agglutinin-120 related to the total Tg(s) in various kinds of thyroid tissue extracts obtained in Example 2.

FIG. 3 shows a ratio (%) of Tg(s) not bound to Concanavalin A related to the total Tg(s) in various kinds of thyroid tissue extracts obtained in Example 3.

FIG. 4 shows a ratio (%) of Tg(s) not bound to Lens culinaris agglutinin related to the total Tg(s) in various kinds of thyroid tissue extracts upon using “anti Tg-86” as an anti Tg antibody, which was obtained in Example 4.

FIG. 5 shows a ratio (%) of Tg(s) not bound to Lens culinaris agglutinin related to the total Tg(s) lectin in various kinds of thyroid tissue extracts upon using “anti Tg-78” as an anti Tg antibody, which was obtained in Example 4.

FIG. 6 shows a ratio (%) of Tg(s) not bound to Concanavalin A related to the total Tg(s)in various kinds of thyroid tissue extracts

### DETAILED DESCRIPTION OF THE INVENTION

The present inventors have extensively studied to solve the above mentioned problems to reach a finding that a total amount of Tg(s) and an amount of Tg(s) having a specific sugar chain structure or that of Tg(s) having a sugar chain structure other than the specific sugar chain structure in a living sample can be measured by using each one or more kinds of “proteins capable of specifically binding to Tg(s)” and “proteins capable of specifically binding to a specific sugar chain structure of Tg(s) having the specific sugar chain structure”, and have continued to study on the basis of this finding to reach an additional finding that the amount of Tg(s) having a specific sugar chain structure and/or that of Tg(s) having a sugar chain structure other than the specific sugar chain structure, or a ratio of Tg(s) having a specific sugar chain structure or that of Tg(s) having a sugar chain structure other than the specific sugar chain structure to total amount of Tg(s) in a living sample can advantageously be used for determining malignant of thyroid tumor, and on the basis of those findings, the present invention has been accomplished.

The “proteins capable of binding to a constant region of Tg(s)” of the present invention (hereinafter abbreviated as “a Tg-binding protein”) includes anti-Tg antibody capable of binding to a constant region of Tg(s), a receptor capable of binding to Tg(s), etc. The protein may be used alone or in a suitable combination of two or more thereof. The constant region of Tg(s) means a region having a structure which is common to all Tg(s) in a living sample. The “Tg-binding protein” of the present invention may have a capability of binding also to a region other than the constant region.

The “Tg-binding protein” includes one having a characteristic of “proteins capable of binding to Tg(s) but not capable of binding to Tg(s) to which the “proteins capable of specifically binding to a specific sugar chain structure of Tg(s) having the specific sugar chain structure” (hereinafter abbreviated as “a protein binding to a specific sugar chain”) is already bound (hereinafter abbreviated as a competitive Tg-binding protein”), one having a characteristic of “a protein capable of binding to all Tg(s) regardless whether a protein binding to a specific sugar chain structure is already bound thereto or not” (hereinafter abbreviated as “a non-competitive Tg-binding protein”).

The anti-Tg antibody capable of binding to a constant region of Tg(s) may be any polyclonal antibodies prepared by immunizing an animal such as horse, sheep, rabbit, goat, rat and mouse with the substance to be measured according to a conventional method described in Tadashi Matsuhashi et al. “Meneki Jikkengaku Nyumon” 2nd. ed., Gakkai-Shuppan Center Ltd., 1981; or any monoclonal antibodies produced by hybridomas obtained by fusing cells from a tumor cell line of mouse with mouse spleen cells previously immunized with the substance to be measured according to a conventional cell fusion method established by G. Köhler and C. Milstein [Nature, 256, 495 (1975)].

The “protein binding to a specific sugar chain structure” of the present invention includes an antibody, a lectin, etc. which are capable of specifically binding to a specific sugar chain structure of Tg(s). More definitely, including an antibody reactive with Lewis type sugar chain such as anti-Le^{a} antibody, anti-Le^{b} antibody, anti-Le^{x} antibody, anti-Le^{y} antibody, anti-S-Le^{a} antibody and other antibodies, a lectin capable of binding to L-fucose such as Lotus tetragonolobus agglutinin, a lectin capable of binding to D-galactose or N-acetyl-D-galactosamine such as Arachis hypogoea agglutinin, soybean agglutinin, Ricinus communis agglutinin and phytohemagglutinin, a lectin capable of binding to D-mannose such as Concanavalin A, Lens culinaris agglutinin and Pisum sativum agglutinin, a lectin capable of binding to N-acetylglucosamine such as wheat germ agglutinin and Datura stramonium agglutinin, a lectin capable of binding to sialic acid such as Limulus polyphemus agglutinin, etc., among which those capable of binding to D-galactose or N-acetyl-D-galagtosamie and those capable of binding to D-mannose are preferable. The lectin may be used alone or in a suitable combination of two or more thereof.

“The capable of binding to a sugar chain structure” in the above classification of the lectin means that a lectin once bound to an affinity column immobilized a suitable sugar chain can be eluted by this sugar. For instance, a lectin capable of binding to D-galactose or N-acetyl-D-galactosamine means that a lectin once bound to an affinity column can be eluted by this D-galactose or N-acetyl-D-galactosamine.

The antibody capable of specifically binding to a specific sugar chain structure of Tg(s) having the specific sugar chain structure may also be polyclonal antibodies or monoclonal antibodies prepared by a conventional manner as mentioned above.

The specific sugar chain structure in the present invention includes particularly (1) a sugar chain structure to which the above mentioned lectin can be bound and (2) a sugar chain structure contained in Tg(s) which is produced by carcinoma cell such as thyroid carcinoma. More particularly, the sugar chain structures described in documents such as Yamamoto, K., Eur. J. Biochem., vol. 143, 133-144, 1984 and so on can be mentioned.

The measurement method of the present invention is characterized in that various kinds of Tg(s) in a sample derived from a living body such as plasma, serum, cerebrospinal fluid, extracted solution of various kinds of living tissues and urine are measured by using “a Tg-binding protein” and “a protein binding to a specific sugar chain structure” in a suitable combination, and the definite object to be measured includes a total Tg(s) amount(s), an amount of Tg(s) having a specific sugar chain structure, an amount of Tg(s) having a sugar chain structure other than the specific sugar chain structure. Tg(s) is decomposed in a living body to give various kinds of fragments, and these fragments to which “a Tg-binding protein” and/or “a protein binding to a specific sugar chain structure” can be bound, among them, can also be the object to be measured in the present invention.

Those objects to be measured can be measured separately or simultaneously in one shot step.

The follows are working modes of the present invention.

(1) A method for measuring Tg(s), which comprises

\mathring{1} reacting a sample originated from a living body with either one of (a) a protein-(1) capable of binding to a constant region of Tg(s) or (b) a protein-(2) capable of specifically binding to a specific sugar chain structure of Tg(s) having the specific sugar chain structure to form a conjugate of [the protein-(1)]-[Tg] or [the protein-(2)]-[Tg],

\mathring{2} reacting the resulting conjugate with the other one of (a) the protein-(1) or (b) the protein-(2), which is labeled by a labeling substance, to form a conjugate of [the protein-(1)]-[Tg]-[the labeled protein-(2)] or [the protein-(2)]-[Tg]-[the labeled protein-(1)],

\mathring{3} separating the conjugate from the free labeled protein (2) or the free labeled protein-(1), and

\mathring{4} measuring an amount of the labeling substance in the conjugate formed in the step \mathring{2}, whereby an amount of a Tg(s) having the specific sugar chain structure is obtained.

(2) A method for measuring Tg(s), which comprises

\mathring{1} reacting a sample originated from a living body with a labeled protein-(2) capable of specifically binding to a specific sugar chain structure of Tg(s) having the specific sugar chain structure and a protein-(3) capable of binding to all Tg(s) regardless whether the labeled protein-(2) is already bound thereto or not to form a conjugate of [the labeled protein-(2)]-[Tg]-[the protein-(3)], and

\mathring{2} separating the conjugate from the free labeled protein-(2), and

\mathring{3} measuring an amount of the labeling substance in the conjugate formed in the step \mathring{1}, whereby an amount of a Tg(s) having the specific sugar chain structure is obtained.

(3) A method for measuring Tg(s), which comprises

\mathring{1} reacting a sample originated from a living body with a protein-(2) capable of specifically binding to a specific sugar chain structure of Tg(s) having the specific sugar chain structure to form a conjugate of [Tg]-[the protein-(2)],

\mathring{2} separating the conjugate from Tg(s) to which the protein-(2) is not bound, and

\mathring{3} measuring an amount of the Tg(s) having the specific sugar chain structure in the conjugate.

(4) A method for measuring Tg(s), which comprises

\mathring{1} reacting a sample originated from a living body with a protein-(1) capable of binding to a constant region of Tg(s) to form a conjugate of [the protein-(1)]-[Tg],

\mathring{2} reacting the conjugate with both of a protein-(2) capable of specifically binding to a specific sugar chain structure of Tg(s) having the specific sugar chain structure, and then reacted with a labeled protein-(4) capable of binding to Tg(s) but not capable of binding to Tg(s) to which the protein-(2) is already bound, to form conjugate (a) of [the protein-(1)]-[Tg]-[the protein-(2)] and a conjugate (b) [the protein-(1)]-[Tg]-[the labeled protein-(4)],

\mathring{3} separating the conjugates from the free labeled protein-(4), and

\mathring{4} measuring an amount of the labeling substance in the conjugate (b) formed in the step \mathring{2}, whereby an amount of Tg(s) having a sugar chain structure other than the specific sugar chain structure is obtained.

(5) A method for measuring Tg(s), which comprises

\mathring{1} reacting a sample originated from a living body with a protein-(2) capable of specifically binding to a specific sugar chain structure of Tg(s) having the specific sugar chain structure, and then reacted with a protein-(4) capable of binding to Tg(s) but not capable of binding to Tg(s) to which the protein-(2) is already bound to form a conjugate of (a) [the protein-(2)]-[Tg] and a conjugate of (b) [the protein-(4)]-[Tg],

\mathring{2} separating the conjugate (b) from others,

\mathring{3} reacting the conjugate (b) with a labeled protein-(1) capable of binding to a constant region of Tg(s) to form a conjugate (c) of [the protein-(4)]-[Tg]-[the labeled protein(1)],

\mathring{4} separating the conjugate (c) from free labeled protein-(1), and

\mathring{5} measuring an amount of the labeling substance in the conjugate (c) formed in the step \mathring{3}, whereby an amount of Tg(s) having a sugar chain structure other than the specific sugar chain structure.

(6) A method for measuring Tg(s), which comprises

\mathring{1} reacting a sample originated from a living body with both a protein-(2) capable of specifically binding to a specific sugar chain structure of Tg(s) having the specific sugar chain structure and a labeled protein-(4) capable of binding to Tg(s) but not capable of specifically binding to a Tg(s) to which the protein-(2) is already bound to form a conjugate of (a) [the protein-(2)]-[Tg] and a conjugate of (b) [the labeled protein-(4)]-[Tg], and

\mathring{2} separating the conjugate (b) from the free labeled protein-(4), and

\mathring{3} measuring an amount of the labeling substance in the conjugate (b) formed in the step \mathring{3}, whereby an amount of Tg(s) having a sugar chain structure other than the specific sugar chain structure is obtained.

(7) A method for measuring Tg(s), which comprises

\mathring{1} reacting sample originated from a living body with both a labeled protein-(1) capable of binding to a constant region of Tg(a) and a protein-(2) capable of specifically binding to a specific sugar chain structure of Tg(s) having the specific sugar chain structure to form conjugates of (a) [the labeled protein-(1)]-[Tg] and (b) [the labeled protein-(l)]-[Tg]-[the protein-(2)], and

\mathring{2} separating the each conjugates from the free labeled protein-(1), and

\mathring{3} measuring the amounts of the labeling substances in each of the conjugate (a) and conjugate (b) formed in the step \mathring{1} are measured respectively, whereby amounts of Tg(s) having the specific sugar chain structure, Tg(s) having a sugar chain structure other than the specific sugar chain structure and total Tg(s) are obtained.

(8) A method for measuring Tg(s), which comprises

\mathring{1} reacting a sample originated from a living body with a protein-(2) capable of specifically binding to a specific sugar chain structure of Tg(s) having the specific sugar chain structure and a labeled protein-(3) capable of binding to all Tg(s) regardless whether the protein-(2) is already bound thereto or not, and a protein-(4) capable of binding to Tg(s) but not capable of binding to Tg(s) to which the protein-(2) is already bound to form conjugates of (a) [the labeled protein-(3)]-[Tg]-[the protein-(4)] and (b) [the labeled protein-(3)]-[Tg]-[the protein-(2)],

\mathring{2} removing the free labeled protein-(3), and separating the conjugates (a) and (b), and

\mathring{3} measuring the amounts of the labeling substances in each conjugate (a) and (b) formed in the step \mathring{1}, whereby amounts of Tg(s) having the specific sugar chain structure, Tg(s) having a sugar chain structure other than the specific sugar chain structure and total Tg(s) are obtained.

(9) A method for measuring Tg(s), which comprises

\mathring{1} reacting a sample originated from a living body with a labeled protein-(1) capable of binding to a constant region of Tg(s) and a protein-(2) capable of specifically binding to a specific sugar chain structure of Tg(s) having the specific sugar chain structure and a protein-(4) capable of binding to Tg(s) but not capable of binding to Tg(s) to which the protein-(2) is already bound to form conjugates of (a) [the labeled protein-(1)]-[Tg]-[the protein-(4)] and [the labeled protein-(1)]-[Tg]-[the protein-(2)]

\mathring{2} removing the free labeled protein-(1), and separating the conjugate (a) and (b), and

\mathring{3} measuring the amounts of the labeling substances in each conjugate (a) and (b) formed in the step \mathring{1}, whereby amounts of Tg(s) having the specific sugar chain structure, Tg(s) having a sugar chain structure other than the specific sugar chain structure and total Tg(s) are obtained.

The following are specific examples of the measurement methods.

I. Methods for Measuring the Objects Separately

The objects to be measured, namely, a total Tg(s) amount, an amount of Tg(s) having a specific sugar chain structure and an amount of Tg(s) having a sugar chain structure other than the specific sugar chain structure are respectively measured by the following.

I-1. Measurement of a Total Tg(s) Amount

It can be measured by a per se known measurement method using “a Tg-binding protein”.

I-2. Measurement of an amount of Tg(s) having a specific sugar chain structure

I-2-1) A Method Using “a Tg-binding Protein” Immobilized On An Insoluble Carrier

A “Tg-binding protein” immobilized on an insoluble carrier is reacted with a sample originated from a living body such as plasma, serum, cerebrospinal fluid, extracted solution of various kinds of living tissues and urine, whereby the following immobilized conjugate is formed.

Insoluble Carrier—“A Tg-binding Protein”—Tg

Then, unnecessary co-existing substances are removed by, for instance, washing, and the immobilized conjugate is reacted further with a “protein binding to a specific sugar chain structure” bound to a labeling substance (hereinafter abbreviated as “a labeled protein binding to a specific sugar chain structure”), whereby the following immobilized conjugate is formed.

Insoluble Carrier—“A Tg-binding Protein”—Tg—“A Labeled Protein Binding To A Specific Sugar Chain Structure

Then, the immobilized conjugate product is, washed, for instance, to remove a free “labeled protein binding to a specific sugar chain structure”, and an amount of the labeling substance in the immobilized conjugate is measured after a suitable measurement method, and the resulting measured value is applied to a calibration curve showing a relationship between an amount of a labeling substance (measured value) and a concentration of Tg(s) which is previously obtained by measuring a standard solution containing a known amount of Tg(s) having a specific sugar chain structure after a similar method, whereby an amount of Tg(s) having a specific sugar chain structure in the sample can be obtained.

I-2-2) A Method Using “A Protein Binding To A Specific Sugar Chain Structure” Immobilized On An Insoluble Carrier

A sample originated from a living body such as plasma, serum, cerebrospinal fluid, extracted solution of various kinds of living tissues and urine is reacted with “a protein binding to a specific sugar chain structure” immobilized on an insoluble carrier, whereby the following conjugate is formed.

Insoluble Carrier—“A Protein Binding To A Specific Sugar Chain Structure”—Tg

Then, unnecessary co-existing substances are removed, for instance, by washing, and further “a Tg-binding protein” bound to a labeling substance (hereinafter abbreviated as “a labeled Tg-binding protein”) is reacted with the conjugate to give the following immobilized conjugate.

Insoluble Carrier—“A Protein Binding To A Specific Sugar Chain Structure”—Tg—“A Labeled Tg-binding Protein”

Then, the immobilized conjugate product is washed, for instance, to remove a free “labeled Tg-binding protein” and an amount of the labeling substance in the immobilized conjugate is measured after a suitable measurement method, and the resulting measured value is applied to a calibration curve showing a relationship between an amount of a labeling substance (measured value) and a concentration of Tg(s) which is previously obtained by measuring a standard solution containing a known amount of Tg(s) having a specific sugar chain structure after a similar method, whereby an amount of Tg(s) having a specific sugar chain structure in the sample can be obtained.

I-2-3) A Method Using “A Labeled Protein Binding To A Specific Sugar Chain Structure” and High-performance Liquid Chromatography (HPLC), etc.

A sample originated from a living body such as plasma, serum, cerebrospinal fluid, extracted solution of various kinds of living tissues and urine is reacted with “a labeled protein binding to a specific sugar chain structure” and “a non-competitive Tg-binding protein” to form the following conjugate in the sample.

“a labeled protein binding to a specific sugar chain structure”-Tg-“a non-competitive Tg-binding protein”

Then, the conjugate and a free “labeled protein binding to a specific sugar chain structure” are separated from each other by HPLC packed with a suitable packing agent, electrophoresis, etc. and an amount of the labeled substance in the conjugate is measured after a suitable method, and the resulting measured value is applied to a calibration curve showing a relationship between an amount of a labeling substance (measured value) and a concentration of Tg(s) which is previously obtained by measuring a standard solution containing a known amount of Tg(s) having a specific sugar chain structure after a similar method, whereby an amount of Tg(s) having a specific sugar chain structure in the sample can be obtained.

I-3. Measurement Of Tg(s) Having A Sugar Chain Structure Other Than The Specific Sugar Chain Structure

I-3-1) A Method Using A Free “Protein Binding To A Specific Sugar Chain Structure”

At first, a sample originated from a living body such as plasma, serum, cerebrospinal fluid, extracted solution of various kinds of living tissues and urine is reacted with “a protein binding to a specific sugar chain structure” to form a conjugate of Tg(s) having a specific sugar chain structure and “a protein binding to a specific sugar chain structure” (hereinafter sometimes abbreviated as a “sugar chain-binding Tg”). Then, a sugar chain-binding Tg and Tg(s) to which a protein binding to a specific sugar chain structure is not bound, in other words, Tg(s) having a sugar chain structure other than the specific sugar chain structure (hereinafter abbreviated as a “non-binding Tg”), are removed from the sample by a per se known separation method such as centrifugation method, gel filtration method, molecular fraction membrane method and electrophoresis method, etc., whereby a sample containing only a non-binding Tg is prepared.

An amount of Tg(s) in thus obtained sample containing only a non-binding Tg is measured by a per se known method using “a Tg-binding protein” to give an amount of a non-binding Tg.

The sample containing only a non-binding Tg may also be prepared by treating the sample with affinity chromatography using “a protein binding to a specific sugar chain structure” immobilized on an insoluble carrier.

I-3-2) A Method Using “A Competitive Tg-binding Protein”

At first, “a Tg-binding protein” immobilized on an insoluble carrier is reacted with a sample originated from a living body such as plasma, serum, cerebrospinal fluid, extracted solution of various kinds of living tissues and urine to form the following immobilized conjugate.

Insoluble Carrier—A Tg-binding Protein”—Tg

Then, unnecessary co-existing substances are removed, for example, by washing, and the immobilized conjugate is reacted with “a protein binding to a specific sugar chain structure” and further with “a competitive Tg-binding protein” to which a labeling substance is bound (hereinafter abbreviated as “a labeled competitive Tg-binding protein”) to give the following immobilized conjugate.

Insoluble Carrier—“A Tg-binding Protein”—Tg—“A Protein Binding To A Specific Sugar Chain Structure”

Insoluble Carrier—“A Tg-binding Protein”—Tg—“A Labeled Competitive Tg-binding Protein”

Then, a free “labeled competitive Tg-binding protein” is removed by, for example, washing the immobilized conjugates, and an amount of the labeled substance in the immobilized conjugates is measured after a suitable method, and the resulting measured value is applied to a calibration curve showing a relationship between an amount of a labeling substance (measured value) and a concentration of Tg(s) which is previously obtained by measuring a standard solution containing a known amount of Tg(s) having a sugar chain structure other than the specific sugar chain structure, namely a non-binding Tg after a similar method, whereby an amount of a non-binding Tg in the sample can be obtained.

I-3-3) A Method Using “A Competitive Tg-binding Protein” Immobilized On An Insoluble Carrier

At first, a sample originated from a living body such as plasma, serum, cerebrospinal fluid, extracted solution of various kinds of living tissues and urine is reacted with “a protein binding to a specific sugar chain structure” to form “a sugar chain-binding Tg” in the sample, and then the sample is reacted with “a competitive Tg-binding protein” immobilized on an insoluble carrier to form the following immobilized conjugate.

Insoluble Carrier—“A Competitive Tg-binding Protein”—A Non-binding Tg

Then, unnecessary co-existing substances are removed, for instance, by washing, and the immobilized conjugate is reacted with “a labeled Tg-binding protein” to form the following immobilized conjugate.

Insoluble Carrier—“A Competitive Tg-binding Protein”—A Non-binding Protein—“A Labeled Tg-binding Protein

Then, a free “labeled Tg-binding protein” is removed by, for instance, washing the immobilized conjugate, and an amount of the labeling substance in the immobilized conjugate is measured after a suitable method, and the resulting measured value is applied to a calibration curve showing a relationship between an amount of a labeling substance (measured value) and a concentration of Tg(s) which is previously obtained by measuring a standard solution containing a known amount of a non-binding Tg after a similar method, whereby an amount of a non-binding Tg in the sample can be obtained.

I-3-4) A method using “a labeled competitive Tg-binding protein” and HPLC, etc.

At first, a sample originated from a living body such as plasma, serum, cerebrospinal fluid, extracted solution of various kinds of living tissues and urine is reacted with “a protein binding to a specific sugar chain structure” to form a sugar chain-binding Tg. Then, the resulting sample is reacted with “a labeled competitive Tg-binding protein” to form the following conjugate.

a non-binding Tg—“a labeled competitive Tg-binding protein”

Then, the conjugate is separated from a free “labeled competitive Tg-binding protein” by HPLC packed with a suitable packing agent, electrophoresis, etc., and an amount of the labeling substance in the conjugate is measured by a suitable method, and the resulting measured value is applied to a calibration curve showing a relationship between an amount of a labeling substance (measured value) and a concentration of Tg(s) which is previously obtained by measuring a standard solution containing a known amount of a non-binding Tg after a similar method, whereby an amount of a non-binding Tg in the sample can be obtained.

Needless to say, an amount of Tg(s) having a specific sugar chain structure (sugar chain-binding Tg) can be obtained by subtracting an amount of Tg(s) having a sugar chain structure other than the specific sugar chain structure (a non-binding Tg) from a total Tg(s) amount, and an amount of Tg(s) having a sugar chain structure other than the specific sugar chain structure (a non-binding Tg) can be obtained by subtracting an amount of Tg(s) having a specific sugar chain structure (a sugar chain-binding Tg) from a total Tg(s) amount.

II. A Method For Measuring The Objects Simultaneously In One Shot Step

II-1. A Method Using “A Labeled Tg-binding Protein” And “A Protein Binding To A Specific Sugar Chain Structure”

This method can be conducted as follows after a method disclosed in Japanese Patent Publication-Kokai-No. 191027/1995.

Namely, at first, \mathring{1} a sample originated from a living body such as plasma, serum, cerebrospinal fluid, extracted solution of various kinds of living tissues and urine is reacted with “a labeled Tg-binding protein” and “a protein binding to a specific sugar chain structure”, or \mathring{2} the sample is reacted with “a labeled Tg-binding protein” and then the resulting reaction solution is further reacted with a protein binding to a specific sugar chain structure” added to the solution, whereby the following conjugates are formed.

“a labeled Tg-binding protein”—Tg

“a labeled Tg-binding protein”—Tg—“a protein binding to a specific sugar chain structure”

Then, the conjugates and a free “labeled Tg-binding protein” are separated from each other by using HPLC packed with a suitable packing agent, electrophoresis, etc. and amounts of the labeled substances in the respective conjugates are measured after a suitable method, and the resulting measured values are applied to a calibration curve showing a relationship between an amount of a labeling substance (measured value) and a concentration of Tg(s) which is previously obtained by measuring standard solutions containing a known amount of Tg(s) having a specific sugar chain structure and/or Tg(s) having a sugar chain structure other than the specific sugar chain structure after a similar method, whereby an amount of Tg(s) having a specific sugar chain structure, an amount of Tg(s) having a sugar chain structure other than a specific sugar chain structure and a total amount thereof, namely a total Tg(s) amount, in the sample can be obtained simultaneously in one step.

As the method for separation of conjugates and a free “labeled Tg-binding protein” from one another, HPLC is preferable because it is easily handled and can be conducted repeatedly.

As the “Tg-binding protein”, a non-competitive one is preferable.

II-2. A Method Using “A Non-competitive Tg-binding Protein”, “A Competitive Tg-binding Protein” And “A Protein Binding To A Specific Sugar Chain Structure”

At first, a sample originated from a living body such as plasma, serum, cerebrospinal fluid, extracted solution of various kinds of living tissues and urine is reacted with “a non-competitive Tg-binding protein” to which a suitable labeling substance is bound (hereinafter abbreviated as “a labeled non-competitive Tg-binding protein”), “a competitive Tg-binding protein” and “a protein binding to a specific sugar chain structure” to form the following conjugates.

“a labeled non-competitive Tg-binding protein”—Tg—“a competitive Tg-binding protein”

“a labeled non-competitive Tg-binding protein”—Tg—“a protein binding to a specific sugar chain structure”

Then, the conjugates and a free “labeled non-competitive Tg-binding protein” are separated from each other by using HPLC packed with a suitable packing agent, electrophoresis, etc. and amounts of the labeled substances in the respective conjugates are measured after a suitable method, and the resulting measured values are applied to a calibration curve showing a relationship between an amount of a labeling substance (measured value) and a concentration of various kinds of Tg(s) which is previously obtained by measuring standard solutions containing a known amount of Tg(s) having a specific sugar chain structure and/or Tg(s) having a sugar chain structure other than the specific sugar chain structure after a similar method, whereby an amount of Tg(s) having a specific sugar chain structure, an amount of Tg(s) having a sugar chain structure other than the specific sugar chain structure and a total amount thereof, namely a total Tg(s) amount, in the sample can be obtained simultaneously in one step. Further, by allowing “a non-competitive Tg-binding protein” having a different epitope from “the labeled non-competitive Tg-binding protein” to take a reaction at the same time, difference between the properties of the conjugates and those of serum ingredients can be increased, as a result of which influences by serum ingredients can be minimized to increase measurement accuracy. In this point of view, this technique is desirable.

As the method for separation of conjugates and a free “labeled non-competitive Tg-binding protein” from one another, HPLC is preferable because it can be conducted repeatedly.

II-3. A Method Using “A Labeled Tg-binding Protein” And “A Competitive Tg-binding Protein”

At first, \mathring{1} a sample originated from a living body such as plasma, serum, cerebrospinal fluid, extracted solution of various kinds of living tissues and urine is reacted with “a labeled Tg-binding protein”, and further “a protein binding to a specific sugar chain structure” and “a competitive Tg-binding protein” are reacted with the above reaction solution to form the following conjugates.

“a labeled Tg-binding protein”—Tg—“a competitive Tg-binding protein”

“a labeled Tg-binding protein”—Tg—“a protein binding to a specific sugar chain structure”

Then, the conjugates and a free “labeled non-competitive Tg-binding protein” are separated from each other by using HPLC packed with a suitable packing agent, electrophoresis, etc. and amounts of the labeled substances in the respective conjugates are measured after a suitable method, and the resulting measured values are applied to a calibration curve showing a relationship between the measured values of a labeling substance and a concentration of various kinds of Tg(s) which is previously obtained by measuring standard solutions containing a known amount of Tg(s) having a specific sugar chain structure and/or Tg(s) having a sugar chain structure other than the specific sugar chain structure after a similar method, whereby an amount of Tg(s) having a specific sugar chain structure, an amount of Tg(s) having a sugar chain structure other than the specific sugar chain structure and a total amount thereof, namely a total Tg(s) amount, in the sample can be obtained simultaneously in one shot step. Further, by allowing “a Tg-binding protein” having a different epitope from “the labeled Tg-binding protein” to take a reaction after the reaction with “a labeled Tg-binding protein”, difference between the properties of the conjugates and those of serum ingredients can be increased, as a result of which influences by serum ingredients can be minimized to increase measurement accuracy. In this point of view, this technique is desirable.

As the method for separation of conjugates and a free “labeled Tg-binding protein” from one another, HPLC is preferable because it is easily handled and can be conducted repeatedly.

The per se known measurement method using “a Tg-binding protein” can be conducted with the use of an anti Tg antibody, for example, as the “Tg-binding protein” after an immunological assay such as enzyme immunoassay (EIA), radioimmunoassay (RIA), enzyme-linked immunosorbent assay (ELISA), fluoroimmunoassay (FIA) and a method using HPLC (Japanese Patent Publication-Kokai-No. 301995/1997), and the measurement mechanism may be any of a sandwich method. a competitive method, a double antibody technique method, etc.

The insoluble carrier used for immobilizing various kinds of “a Tg-binding protein” and “a protein binding to a specific sugar chain structure” may be any of those so far used in the field of the above immunological assay and includes beads, tube, a special tray or microtiter plate in which many tubes are molded in a body, etc., which are made from metal, glass, ceramic, silicone rubber, synthetic polymer such as polystyrene, polyvinyl chloride, polypropylene, acryl resin and polymethylmethacrylate, etc., and an immobilizing method may be any of so far used in the field of immunological assay, such as physical adsorption methods and chemical binding methods.

The labeling substance to be bound to “the Tg-binding protein” and “the protein binding to a specific sugar chain structure” is bound includes alkali phosphatase, β-galactosidase, peroxidase, microperoxidase, glucoseoxidase, glucose-6-phosphate dehydrogenase, acetylcholinesterase, malic dehydrogenase, luciferase and other enzymes which are used in EIA, ^{99m}Tc, ¹³¹I, ¹²⁵I, ¹⁴C, ³H and other radioisotopes which are used in RIA, fluoresceine, dansyl, fluorescamine, coumarin, naphthylamine and their derivatives and other fluorescent substances which are used in FIA, luciferin, isoluminol, luminol, bis(2,4,6-trifluorophenyl)oxalate and other luminescent substances, phenol, naphthol, anthracene and their derivatives and other substances which can absorb an ultarviolet light, 4-amino-2,2,6,6-tetramethylpiperidine-1-oxyl, 3-amino-2,2,5,5-tetramethylpyrrolidine-1-oxyl, 2,6-di-t-butyl-α-(3,5-di-t-butyl-4-oxo-2,5-cyclohexadien-1-ylidene)-p-tolyl oxyl and other oxyl group-containing compounds which have characteristics as spin-labeling agent.

The method for binding (labeling) the above labeling substance to “the Tg-binding protein” and “the protein binding to a specific sugar chain structure” is bound may be conducted after a per se known one so far conducted generally in per se known EIA, RIA, FIA, etc. As the labeling method, a conventional one using a reaction of avidin (or streptoavidin) with biotin may be used.

HPLC apparatus used in the measurement method using HPLC in the present invention may be any one so far used in this kind of field.

In the measurement method using HPLC of the present invention, for the purpose of clearer separation of a conjugate from a free “labeled Tg-binding protein” (or “labeled protein binding to a specific sugar chain structure”), use may be made of “the Tg-binding protein” or “the protein binding to a specific sugar chain structure” is bound, to which a substance enhancing the separation (hereinafter abbreviated as “separation enhancing substance”) disclosed in Japanese Patent Publication-Kokai-No. 191027/1995, No. 301995/1997, etc. is may be used.

The “separation enhancing substance” used for this purpose includes preferably proteins such as α-chymotripsinogen, β-galactosidase, lysozyme, cytochrome c and trypsin inhibitor; peptides containing an amino acid such as phenylalanin, proline, arginine, ricin, aspartic acid and glutamic acid; halogen atoms such as bromine, chlorine and iodine; synthetic polymers such as polyethylene glycol; polyamino acids such as polyglutamic acid, polyaspartic acid, polylysin, polyarginine, polyphenylalanin and polytyrosine; alkyl chains having 3 to 10 carbon atoms; fatty acids such as palmitic acid, oleic acid and stearic acid; chemical substances having a group reactive with “the Tg-binding protein” and “the protein binding to a specific sugar chain structure” is bound and showing hydrophobic property, such as N-(ε-maleimidocaproyloxy) succinimide (EMCS), N-succinimidyl-6-maleimidohexanoate, Bismaleimidohexane (BMH) and octylamine, peptides containing a strong acid residue such as 4-(p-maleimidophenyl) butylyl Ala-(Tyr(SO₃H))₅ and 4-(p-maleimidophenyl) butylyl Ala-(Tyr(SO₃H))₈ which are disclosed in Japanese Patent Publication-Kokai-No. 301995/1997, etc. The “separation enhancing substance” to be used can be selected considering characteristics (such as pH stability, hydrophobicity, solubility in water and isoelectric point) of the objects to be measured such as Tg(s), Tg-binding proteins and proteins to which a specific sugar chain is bound.

A method for binding “the separation enhancing substance” with “the Tg-binding proteins” and/or “the proteins binding to a specific sugar chain structure” can be conducted after (1) a per se known manner for binding a labeled substance with an antibody generally used in per se known EIA, RIA or FIA (e.g. Yuichi Yamamura “Ikagaku Jikken Koza Vol. 8” 1st ed., NAKAYAMA-SHOTEN Ltd., 1971, Akira Kawano “Zusetsu Keikokotai” 1st ed., Soft Science, Inc., 1983; and Eiji Ishikawa, Tadashi Kawai and Kiyoshi Miyai “Koso Men-eki Sokuteiho” 2nd. ed., IGAKU-SHOIN Ltd., 1982), (2) a per se known manner for modification and binding of a substance (e.g. Ikuzo Uritani, Kensuke Shimura, Michinori Nakamura and Masaru Funazu “Tanpakushitu-no Kagakushiushoku <Jo>, <Ge>” 1st ed., GAKKAO-SHUPPAN CENTER Ltd., 1981; Yuji Inada et al. “Poly (ethylene glycol) Shushoku Tanpakushitsu” Seikagaku vol. 62, No. 11, pp. 1351-1362, Japanese Biochemical Association, 1990; and George H K. and Mark M. M. “DNA PROBES” STOCKTON PRESS, 1989), etc.

By a suitable combination of values of a total Tg(s) amount, an amount of Tg(s) having a specific sugar chain structure and Tg(s) having a sugar chain structure other than the specific sugar chain structure, which are obtained by the measurement method of the present invention, malignancy of thyroid tumor can be determined.

Namely, for instance, by obtaining a content of Tg(s) having a specific sugar chain structure or Tg(s) having a sugar chain structure other than the specific sugar chain structure in the total Tg(s), malignancy of thyroid tumor, in other words, as to whether the tumor is benign or malignant thyroid carcinoma, can be determined.

Further in detail, by obtaining a content of Tg(s) having a specific sugar chain structure or Tg(s) having a sugar chain structure other than the specific sugar chain structure in the total Tg(s) with the use of lectins capable of binding to D-galactose or N-acetyl-D-galactosamine such as Arachis hypogoea agglutinin, soybean agglutinin, Ricinus communis agglutinin and phytohemagglutinin, lectins capable of binding to D-mannose such as concanavalin A, Lens culinaris agglutinin and Pisum sativum agglutinin, etc., as the “protein binding to a specific sugar chain structure” , differentiation of benign thyroid adenoma or Graves' disease, etc. from papillary carcinoma etc., and differentiation of follicular adenoma from follicular carcinoma, etc. becomes possible on the basis of the result thus obtained.

For instance, diagnosis using the extract from a tissue sample can be performed, taking the following phenomenon into consideration;

(1) the ratio (%) of Tg(s) not bound to Con A related to the amount of total Tg(s) in sample from the papillary carcinoma is significantly higher than those in the sample from benign disease such as benign thyroid adenoma, Grave's disease, follicular adenoma or thyroid adenomatous.

(2) the ratio (%) of Tg(s) not bound to Con A, related to the amount of total Tg(s) in sample from the follicular carcinoma is significantly higher than those in the sample from follicular adenoma and normal.

(3) the ratio (%) of Tg(s) not bound to RCA120 related to the amount of total Tg(s) in sample from the papillary carcinoma is significantly higher than those in the sample from Grave's disease and normal.

(4) the ratio (%) of Tg(s) not bound to LCA related to the amount of total Tg(s) in sample from the papillary carcinoma is significantly higher than those in the sample from benign disease such as follicular adenoma, thyroid adnomatous, Grave's disease.

Namely, the method of determining malignancy of thyroid tumor of the present invention has been accomplished on the basis of those facts which have been found for the first time by the present inventors.

The reagent for measurement of Tg(s) of the present invention comprises each one or more of “proteins capable of binding to a constant region of Tg(s)” and “proteins capable of specifically binding to a specific sugar chain structure of Tg(s) having the specific sugar chain structure”, and the preferable embodiments and specific examples thereof are as mentioned above.

The reagent for determination of malignancy of thyroid adenoma comprises each one or more of “proteins capable of binding to a constant region of Tg(s)” and “proteins capable of specifically binding to a specific sugar chain structure of Tg(s) having the specific sugar chain structure”, and the preferable embodiments and specific examples thereof are as mentioned above.

In the reagents, there may be incorporated reagents having generally been used in this field so far as not interfering stability of co-existing reagents and not interfering the reaction of Tg(s) with “Tg-binding proteins” and/or “proteins binding to a specific sugar chain structure”, such as a buffering agent, a reaction accelerator and a stabilizer such as a sugar, a protein, a salt and a surfactant, antiseptic, etc., and their contents may be selected from a range having been generally used in this field.

Further, a metal ion such as magnesium has been known as not influencing upon activity and stability of lectin, and thus the metal ion may also be incorporated in the reagents.

The buffering agent usable in the reagents of the present invention includes all ones having been generally used in immunoturbidmetry, immunonephelometry, RIA, EIA, etc. such as Tris buffer, phosphate buffer, veronal buffer, borate buffer and Good's buffer, and pH upon measurement is not specifically limited so far as an antibody-antigen reaction, a reaction of Tg(s) with lectin, etc. are not restrained and generally 6 to 10.

In the following, the present invention is further explained in detail, and the present invention is not limited thereto by any means.

### EXAMPLE 1

### A Method for Differentiation of Papillary Tumors Using Concanavalin A (ConA)

(Peroxidase Labeled Anti Tg Antibody)

An anti Tg antibody (hereinafter abbreviated as “anti Tg-1”) was bound to peroxidase after a conventional manner to give peroxidase labeled anti Tg antibody (hereinafter abbreviated as “anti Tg-1.POD”).

(Sample)

Human thyroid tissue pieces in an amount of 0.1 g (wet wt) were homogenized in 1 ml of 0.1 M phosphate buffer solution (pH 7.5, containing 0.9% NaCl, hereinafter abbreviated as “PBS”) by a homogenizer, followed by centrifuging at 4° C. at 30000 g for 5 minutes, and thus obtained supernatant was used as the sample.

The human thyroid tissues used were 11 papillary carcinoma specimens, 5 benign thyroid adenoma specimens, 5 Graves' disease specimens and 5 normal thyroid tissue specimens.

(Reagent 1)

Con A (manufactured and sold by Honen Corp.) was dissolved in PBS to give 15 mg/ml solution, which was used as Reagent 1.

(Pre-treatment of Sample)

Each 50 μl of Sample and Reagent 1 were mixed with each other, incubated at 4° C. for overnight and centrifuged at 4° C. at 3000 g for 20 min. to remove precipitates, and the resulting supernatant was used as Pre-treatment solution 1. By the way, Tg(s) bound to Con A was removed by the centrifugation.

PBS was used in place of Reagent 1 after a similar manner to give supernatant which was used as Pre-treatment solution 2.

(Measurement of Tg Content)

PBS in an amount of 1 ml containing 5 μg of an anti Tg antibody having an epitope different from that of “anti Tg-1” (hereinafter abbreviated as “anti Tg-2”) was charged in each of 96 wells of a microplate, followed by keeping standing at 20° C. for 1 hour and washing with PBS, whereby “anti Tg-2” was immobilized. Then, 0.2 ml of PBS containing 1% of fetal bovine serum was added to each of the wells, followed by keeping standing at 20° C. for 1 hour and washing with PBS, whereby blocking treatment was attained. PBS in an amount of 100 μl containing 1% of fetal bovine serum and 50 μl of “Pre-treatment solution 1” or “Pre-treatment solution 2” were charged to each of the wells, followed by allowing a reaction to take place at 20° C. for 1 hour. After the reaction, each of the wells was washed with PBS, and 100 μl of “anti Tg-1.POD” diluted 10000 times was added thereto, followed by allowing a reaction to take place at 20° C. for 1 hour. After the reaction, the wells were washed with PBS and 100 μl of 3,3′,5,5′-tetramethylbenzidine solution (manufactured and sold by Kirkegaad and Perry Labs. Inc.) was added thereto, followed by allowing a reaction to take place at 20° C. for 30 minutes and terminating the reaction by addition of 50 μl of 1 M phosphoric acid. Absorbances at 450 nm of each of wells were measured by Microplate reader Spectra 1 (manufactured and sold by Wako Pure Chemical Industries, Ltd.) and the measured absorbances were applied to a calibration curve showing a relationship between Tg(s) content and absorbance which was previously prepared by using a Tg(s) solution containing a known amount of Tg(s) after a similar manner, whereby Tg(s) contents in “Pre-treating solution 1” and “Pre-treating solution 2”.

Tg(s) content in “Pre-treating solution 1” shows a content of Tg(s) not bound to Con A, and Tg(s) content in “Pre-treating solution 2” shows a total Tg(s) content.

(Calculation of a Ratio of Tg(s) Not Bound to Con A)

A ratio (%) of Tg(s) not bound to Con A is calculated by the following equation.

Ratio (%) of Tg(s) not bound to Con A =[(an amount of Tg(s) not bound to Con A)/(total Tg(s))]×100

(Result)

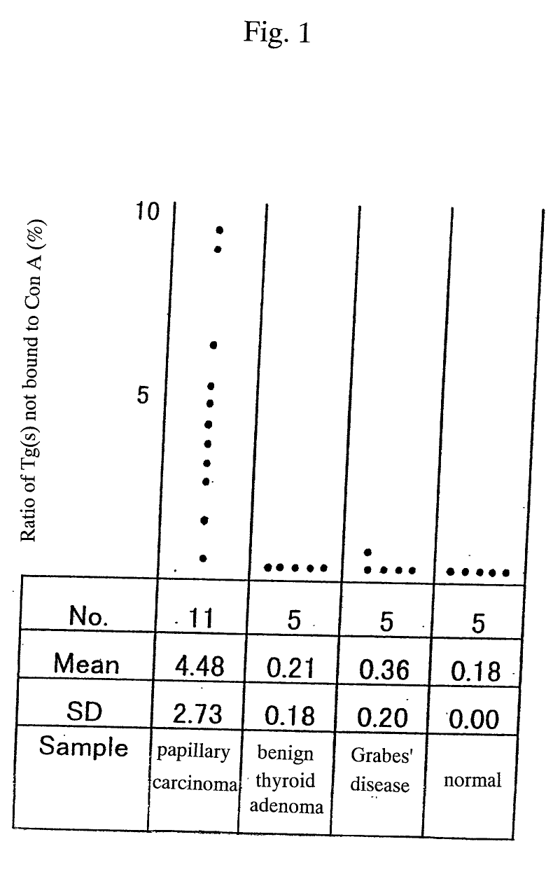

Thus obtained ratio (%) of Tg(s) not bound to Con A is shown in FIG. 1.

As is clear from FIG. 1, a ratio (%) of Tg(s) not bound to Con A in the extract from tissue of papillary carcinoma is significantly higher than those in the extracts from benign thyroid adenoma tissue, Graves' disease tissue and normal thyroid tissue. It is also understood that no difference in a ratio (%) of Tg(s) not bound to Con A is found among benign thyroid adenoma tissue extract, Graves' disease tissue extract and normal thyroid tissue extract. Namely, it can be recognized that an amount of Tg whose sugar chain is changed, in other words, Tg(s) not capable of binding to Con A is increased in papillary carcinoma tissue, and thus a ratio (%) of Tg(s) not bound to Con A is very useful for differentiation of benign thyroid adenoma from papillary carcinoma.

### EXAMPLE 2

### A Method for Differentiation of Papillary Carcinoma Using Ricinus communes Agglutinin-120 (RCA120)

(Peroxidase Labeled Anti Tg Antibody)

Same as Example 1

(Sample)

Sample was prepared by the same manner as in Example 1. The human thyroid tissues used were 7 papillary carcinoma specimens, 5 Graves' disease specimens and 4 normal thyroid tissue specimens.

(Reagent 1)

RCA120 (manufactured and sold by Honen Corp.) was dissolved in PBS to give 2.5 mg/ml solution, which was used as Reagent 1.

(Pre-treatment of Sample)

Pre-treatment solution 1 and 2 were prepared by the same manner as in Example 1, except for using Reagent 1 containing RCA 120 instead of Reagent 1 containing ConA.

(Measurement of Tg Content)

Measurement of Tg(s) content was conducted after the same manner as in Example 1.

Tg(s) content in Pre-treatment solution 1 shows a content of Tg(s) not bound to RCA120 and that in Pre-treatment solution 2 shows a total Tg(s) content.

(Calculation Of A Ratio Of Tg(s) Not Bound to RCA120)

A ratio (%) of Tg(s) not bound to RCA120 is calculated by the following equation.

Ratio (%) of Tg(s) not bound to RCA120=[(an amount of Tg(s) not bound to RCA120/(total Tg(s))]×100

(Result)

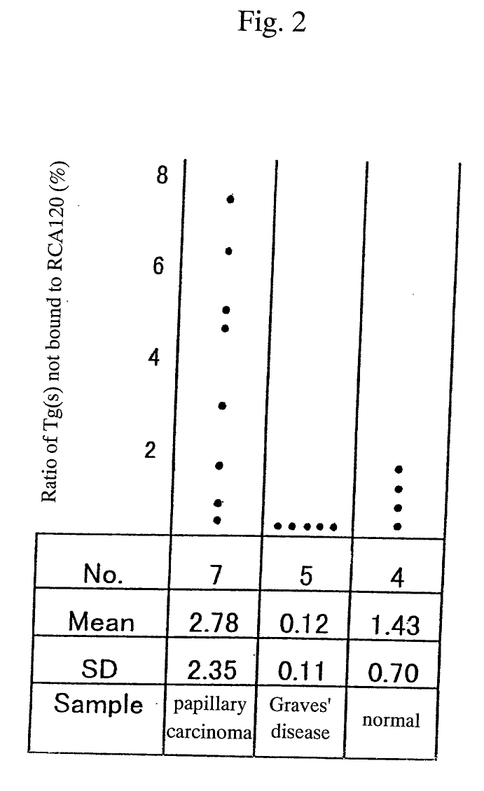

Thus obtained ratio (%) of Tg(s) not bound to RCA120 is shown in FIG. 2.

As is clear from FIG. 2, a ratio (%) of Tg(s) not bound to RCA120 in the extract of tissue of papillary carcinoma is significantly higher than those in the extracts of benign goiter tissue, Graves' disease tissue and normal thyroid tissue. Namely, it can be recognized that an amount of Tg(s) whose sugar chain is changed, in other words, Tg(s) not capable of binding to RCA120 is increased in papillary carcinoma tissue, and thus a ratio (%) of Tg(s) not bound to RCA120 is very useful for differentiation of papillary carcinoma.

### EXAMPLE 3

### Differentiation of Follicular Carcinoma from Follicular Adenoma Using Con A

(Peroxidase Labeled Anti Tg Antibody)

Same as in Example 1

(Sample)

Sample was prepared by the same manner as in Example 1. The human thyroid tissues used were 4 follicular carcinoma specimens, 7 follicular adenoma specimens and 5 normal thyroid tissue specimens.

(Reagent 1)

Same as in Example 1

(Pre-treatment Of Sample)

Pre-treatment solution 1 and 2 were prepared by the same manner as in Example 1.

(Measurement Of Tg(s) Content)

Measurement of Tg(s) content was conducted by the same manner as in Example 1.

Tg(s) content in Pre-treatment solution 1 shows a content of Tg(s) not bound to Con A and that in Pre-treatment solution 2 shows a total Tg(s) amount.

(Calculation Of A Ratio Of Tg(s) Not Bound To Con A)

A ratio (%) of Tg(s) not bound to Con A was calculated by the same manner as in Example 1.

(Result)

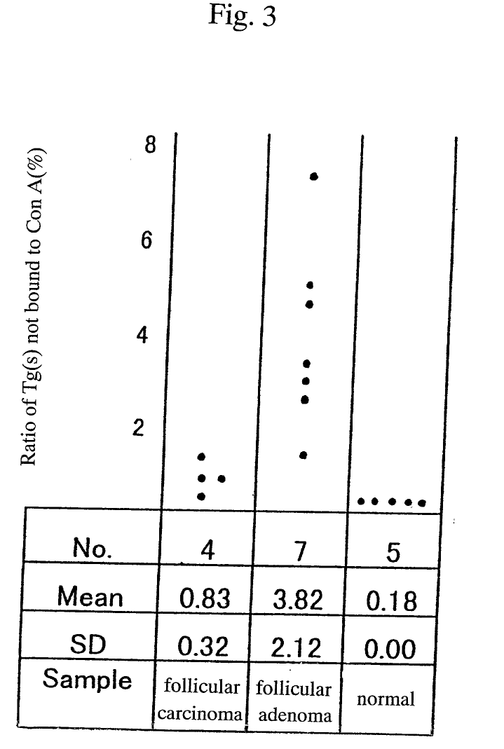

Thus obtained ratio (%) of Tg(s) not bound to Con A is shown in FIG. 3.

As is clear from FIG. 3, a ratio (%) of Tg(s) not bound to Con A becomes higher and higher from a normal thyroid tissue extract, then a follicular carcinoma tissue extract, finally to a follicular adenoma tissue extract in this order, and it can be understood that there is found a significant difference in the ratio between a follicular carcinoma tissue extract and a follicular adenoma tissue extract. From this result, it can be recognized that differentiation between follicular carcinoma and follicular adenoma, which has been difficult even by cytodiagnosis, can easily be attained by measuring a ratio (%) of Tg(s) not bound to Con A.

### EXAMPLE 4

### Fractional Measurement of Tg(s) Having Different Sugar Chain Structures Using Lens culinaris Agglutinin (LCA)

(Peroxidase Labeled Anti Tg Antibody Fab′ Fragment)

Two kinds of anti Tg antibodies having different recognizable epitopes from each other, which do not bind to Tg bound to LCA (hereinafter abbreviated as “anti Tg-86” and “anti Tg-78”, respectively), were treated after a conventional manner to give Fab′ fragments and then they were bound to peroxidase (manufactured and sold by Toyobo Co., Ltd.) after a conventional manner to give peroxidase labeled anti Tg antibody Fab′ fragments (hereinafter abbreviated as “anti Tg-86.Fab′-POD”, “anti Tg-78.Fab′-POD”, respectively).

(Antibody Solution 1)

A 50 mM phosphate buffer solution (pH 7.5, containing 0.15M NaCl and 0.5 (w/v) % bovine serum albumin) containing 2 nM of “anti Tg-86.Fab′-POD” was prepared, which was used as Antibody solution 1.

(Antibody Solution 2)

A 50 mM phosphate buffer solution (pH 7.5, containing 0.15M NaCl and 0.5 (w/v) % bovine serum albumin) containing 3 nM of “anti Tg-78.Fab′-POD” was prepared, which was used as Antibody solution 2.

(Reagent 1)

A 50 mM phosphate buffer solution (pH 7.5, 0.15 M NaCl) containing 15 μM of LCA (manufactured and sold by Honen Corp.) was prepared, which was used as Reagent 1.

(Sample)

Human thyroid tissue pieces in an amount of 0.1 g (wet wt) were homogenized in 1 ml of 0.1 M phosphate buffer (pH 7.2, containing 0.9% of NaCl) using a homogenizer, followed by centrifuging at 4° C. at 100000 g for 60 minutes, and the supernatant obtained was diluted 200 to 1100 times with 50 mM phosphate buffer (pH 7.5, containing 0.15 M NaCl and 0.5 (w/v) % of bovine serum albumin) to give the sample.

The human thyroid tissues used were 4 benign disease specimens (1 follicular adenoma specimen, 2 adenomotous specimens and 1 Graves' disease) and 4 papillary carcinoma specimens.

(Measurement Method)

Sample in an amount of 25 μl was mixed with 15 μl of Reagent 1, followed by allowing a reaction to take place at 8° C. for 1 hour. The reaction solution in an amount of 15 μl was mixed with 90 μl of Antibody solution 1, followed by allowing a reaction to take place further at 8° C. for 30 minutes. The resulting reaction solution in an amount of 50 μl was analyzed using high-performance liquid chromatography (HPLC) under the conditions as mentioned below to measure an amount of Tg(s) not bound to LCA. The same measurement for the total Tg(s) amount was conducted on the same sample using 50 mM of phosphate buffer (pH 7.5, containing 0.15M NaCl and 0.5 (w/v) % bovine serum albumin) in place of Reagent 1 containing LCA. Those two results were applied to the following equation to calculate a ratio (%) of Tg(s) not bound to LCA upon using “anti Tg-86.Fab′-POD”.

A ratio (%) of Tg(s) not bound to LCA =[(an amount of Tg(s) not bound to LCA)/(total Tg(s))]×100

A ratio (%) of Tg(s) not bound to LCA upon using “anti Tg-78.Fab′-POD” was also calculated by the same manner except for using Antibody solution 2 in place of Antibody solution 1.

(HPLC Conditions)

Column: Wakopack Wakosil®-5Diol-200 8.0 mm×300 mm (w)

Eluent: 50 mM phosphate buffer, pH 7.5, 0.15 M NaCl

Substrate solution: 15 mM citrate buffer (pH 5.5, 313 mM acetoaminophenol (manufactured and sold by Dojin Laboratories, containing 0.12% H₂O₂)

Flow rate: the eluent 1.0 mL/min, the substrate solution 0.1 mL/min

Detection: Ex 328 nm, Em 432 nm

(Result)

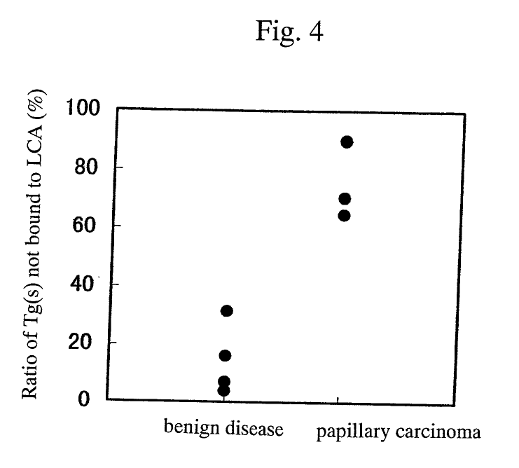

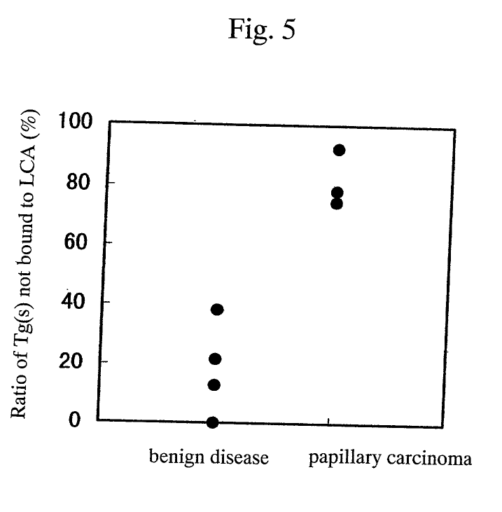

Ratios (%) of Tg(s) not bound to LCA upon using “anti Tg-86.Fab′-POD” and “anti Tg-78.Fab′-POD” are shown in FIG. 4 and FIG. 5, respectively.

It is understood from FIG. 4 and FIG. 5 that the ratio (%) of Tg(s) not bound to LCA in a papillary carcinoma tissue extract is significantly higher than that in a benign disease tissue extract.

Namely, it is understood that differentiation diagnosis of papillary carcinoma from benign disease becomes possible by using LCA.

### Example 5

### Fractional Measurement of Tg(s) Having Different Sugar Chain Structures Using Con A

(Peroxidase Labeled Anti Tg Antibody Fab′ Fragment)

“Anti Tg-86.Fab′-POD” prepared in Example 4 was used.

(Antibody Solution 1)

A 50 mM phosphate buffer solution (pH 7.5, containing 0.15 M NaCl and 0.5 (w/v) % bovine serum albumin) containing 4 nM of “anti Tg-86.Fab′-POD” was prepared which was used as Antibody solution 1.

(Reagent 1)

A 50 mM phosphate buffer solution pH 7.5, 0.15 M NaCl, containing 15 μM of Con A (manufactured and sold by Honen Corp.) was prepared which was used as Reagent 1.

(Sample)

The same one as in Example 4 was used.

(Measurement Method)

Sample in an amount of 10 μl was mixed with 45 μl of Reagent 1, followed by allowing a reaction to take place at 8° C. for 1 hour. The reaction solution was mixed with 45 μl of Antibody solution 1, following by allowing a reaction to take place further at 8° C. for 30 minutes. The resulting reaction solution in an amount of 50 μl was analyzed using high-performance liquid chromatography (HPLC) under the conditions as mentioned below to measure an amount of Tg(s) not bound to Con A. The same measurement for the total Tg(s) amount was conducted on the same sample using 50 mM of phosphate buffer (pH 7.5, containing 0.15M NaCl and 0.5 (w/v) % bovine serum albumin) in place of Reagent 1. Those two results were applied to the following equation to calculate a ratio (%) of Tg(s) not bound to Con A.

A ratio (%) of Tg(s) not bound to Con A =[(an amount of Tg(s) not bound to Con A)/(total Tg(s))]×100

(HPLC Conditions)

Same as in Example 4

(Result)

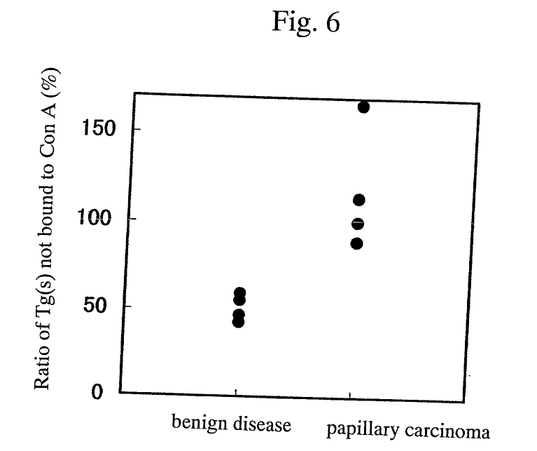

The obtained ratio (%) of Tg(s) not bound to Con A is shown in FIG. 6.

It is understood from FIG. 6 that a ratio of Tg(s) not bound to Con A in papillary carcinoma tissue extract is significantly higher than that in a benign disease tissue extract.

Namely, it is understood that differentiation diagnosis of papillary carcinoma from benign disease becomes possible by using Con A.

The present invention is to provide a method for measuring simply and at high accuracy various kinds of Tg(s) in various kinds living samples and a reagent for this method, and differentiation of papillary carcinoma from benign thyroid adenoma and differentiation of follicular carcinoma from follicular adenoma can be conducted by using a suitable combination of the measurement results of various kinds of Tg(s).

What is claimed is:

1. A method for measuring thyroglobulin(s), comprising using each one or more kinds of proteins capable of binding to a constant region of thyroglobulin(s) and proteins capable of specifically binding to a specific sugar chain structure of thyroglobulin(s) having the specific sugar chain structure.

2. The method according to claim 1, wherein the thyroglobulin(s) to be measured are a total thyroglobulin(s), a thyroglobulin having a specific sugar chain structure and/or a thyroglobulin having a sugar chain structure other than the specific sugar chain structure.

3. The method according to claim 1, wherein the specific sugar chain structure is a sugar chain structure capable of binding to a lectin.

4. The method according to claim 3, wherein the lectin is one capable of binding to D-galactose or N-acetyl-D-galactosamine, or binding to D-mannose.

5. The method according to claim 3, wherein the lectin is Concanavalin A, Lens culinaris agglutinin or Ricinus communis agglutinin.

6. A reagent for measuring thyroglobulin(s), which comprises each one or more kinds of proteins capable of binding to a constant region of thyroglobulin(s) and proteins capable of specifically binding to a specific sugar chain structure of thyroglobulin(s) having the specific sugar chain structure.

7. A reagent for determining malignancy of thyroid tumor, which comprises each one or more kinds of proteins capable of binding to a constant region of thyroglobulin(s) and proteins capable of specifically binding to a specific sugar chain structure of thyroglobulin(s) having the specific sugar chain structure.

8. A method of determining a malignancy of thyroid tumor, which comprises measuring one or more of thyroglobulins having a specific sugar chain structures present in a sample originated from a living body and determining the malignancy of thyroid tumor on the basis of the amounts of the thyroglobulins.

9. The method according to claim 8, wherein the malignancy of thyroid tumor is determined on the basis of the proportion of one or more of thyroglobulins having a specific sugar chain structure, related to the total amount of thyroglobulin(s).

10. The method according to claim 8, wherein the measurement of thyroglobulin(s) is done after one or more of thyroglobulins are separated from one another on the basis of the difference between or among their sugar chain structure.

11. The method according to claim 10, wherein the separation is performed by using each one or more kinds of proteins capable of binding to a constant region of thyroglobulin(s) and proteins capable of specifically binding to a specific sugar chain structure of thyroglobulin(s) having the specific sugar chain structure.

12. The method according to claim 8, wherein the specific sugar chain structure is a sugar chain structure capable of binding to a lectin.

13. The method according to claim 12, wherein the lectin is one capable of binding to D-galactose or N-acetyl-D-galactosamine, or binding to D-mannose.

14. The method according to claim 12, wherein the lectin is Concanavalin A, Lens culinaris agglutinin or Ricinus communis agglutinin.

15. The method according to claim 8, wherein the specific sugar chain structure is one found in thyroglobulin(s) which is produced by a carcinoma cell.

16. The method as claimed in claim 15, wherein the carcinoma cell is originated from thyroid carcinoma.

17. A method of determining a malignancy of thyroid tumor, which comprises separating thyroglobulins from one another present in a sample originated from a living body on the basis of the difference between or among their sugar chain structures, measuring the proportion of one or more of thyroglobulin(s) having a specific sugar chain structure, related to the total amount of thyroglobulin(s), and determining the malignancy of thyroid tumor on the basis of a result of the measurement.

18. The method according to claim 17, wherein the separation is performed by using each one or more kinds of proteins capable of binding to a constant region of thyroglobulin(s) and proteins capable of specifically binding to a specific sugar chain structure of thyroglobulin(s) having a specific sugar chain structure.
